(19) United States
(12) Patent Application Publication
(10) Pub. No.: US 2018/0271417 A1
(43) Pub. Date: Sep. 27, 2018

(54) METHOD AND A DEVICE FOR NON-INVASIVE MONITORING OF A BLOOD GLUCOSE LEVEL OF A USER

(71) Applicant: Wipro Limited, Bangalore (IN)

(72) Inventors: Vinod Pathangay, Bangalore (IN); Anandaraj Thangappan, Bangalore (IN)

(21) Appl. No.: 15/468,910

(22) Filed: Mar. 24, 2017

(30) Foreign Application Priority Data
Mar. 21, 2017 (IN) 201741009909

(51) Int. Cl. A61B 5/145; A61B 5/01; A61B 5/00; A61B 5/053

(52) U.S. Cl. CPC A61B 5/14532; A61B 5/01; A61B 5/0059; A61B 5/681; A61B 5/74; A61B 5/0002; A61B 5/053

(57) ABSTRACT

A method and a device are described for non-invasive monitoring of blood glucose level of a user. The method includes determining an electrical skin impedance between a first point and a second point of a surface of skin of user using a skin impedance sensor. In an embodiment, electrical skin impedance is indicative of an opacity of surface of skin between first point and second point. The method includes determining a temperature and a hyper spectral signature of skin of user using a temperature sensor and a hyperspectral sensor. The method includes updating a light intensity of a light source based on temperature and hyperspectral signature. In an embodiment, surface of the skin is illuminated based on updated light intensity of light source. The method includes computing a blood glucose level using temperature, hyper spectral signature, and electrical skin impedance. The method includes providing computed blood glucose level to user.

This application claims the benefit of Indian Patent Application Serial No. 201741009909, filed Mar. 21, 2017, which is hereby incorporated by reference in its entirety.

### TECHNICAL FIELD

The present subject matter is related, in general to monitoring blood glucose levels and more specifically, but not exclusively to a method and a device for non-invasive monitoring of blood glucose level of a user.

### BACKGROUND

Diagnosis of diseases has always been regarded as a primary cardinal step in any medical issue. A healthcare diagnosis is a key element in measuring the current health condition of a patient or a human being, to proactively take precautionary measures that may be helpful for the patient. Over a period of time may of these diagnostic procedures are evolved from being invasive to non-invasive due to the ensued convenience of patients. One such example is diagnosis of blood sugar levels in humans. Conventionally blood sugar detection method has been an invasive step. To simplify, it involves use of a disposable injection to suck the blood and then test for blood sugar levels in a lab. The existing methods are mostly based on pricking of the skin in order to extract few droplets of blood for chemical analysis. This can be traumatic for some users and there is also a risk of infection caused by inadvertent reuse of needles.

Moreover, the non-invasive methods which are available to detect blood glucose are not accurate to perform spectral analysis of blood. The available non-invasive methods suffer from impediments as they cannot handle various skin types and skin thickness, in order to effectively monitor blood glucose level in the blood.

Please establish the problem of non-invasive monitoring devices. Mention about change in spectral signature, thickness/opacity of skin and thus illumination of skin with pre-defined light may lead to false positives of blood glucose level of a user. Elaborate on this aspect in the background.

The limitations and disadvantages of conventional and traditional approaches may become apparent to one skilled in the art, through comparison of systems described with some aspects of the present disclosure, as set forth in the remainder of the present application and with reference to the drawings.

### SUMMARY

The foregoing summary is illustrative only and is not intended to be in any way limiting. In addition to the illustrative aspects, embodiments, and features described above, further aspects, embodiments, and features will become apparent by reference to the drawings and the following detailed description.

According to embodiments illustrated herein, there may be provided a method for non-invasive monitoring of a blood glucose level of a user. The method may include determining an electrical skin impedance between a first point and a second point of a surface of skin of the user using a skin impedance sensor. In an embodiment, the electrical skin impedance may be indicative of an opacity of the surface of the skin between the first point and the second point. The method may determine a temperature and a hyper spectral signature of the skin of the user using a temperature sensor and a hyperspectral sensor. In an embodiment, the method may update a light intensity of a light source based on the temperature and the hyperspectral signature. In an embodiment, the surface of the skin may be illuminated based on the updated light intensity of the light source. The method may include computing a blood glucose level using the temperature, the hyper spectral signature, and the electrical skin impedance. In an embodiment, the method may provide the computed blood glucose level to the user.

According to embodiments illustrated herein, there may be provided a glucose monitoring device to monitor a blood glucose level of a user, the glucose monitoring device, which may include a processor and a memory communicatively coupled to the processor. In an embodiment, the memory stores processor instructions, which, on execution, causes the processor to determine an electrical skin impedance between a first point and a second point of a surface of skin of the user using a skin impedance sensor. In an embodiment, the electrical skin impedance may be indicative of an opacity of the surface of the skin between the first point and the second point. The processor may be configured to determine a temperature and a hyper spectral signature of the skin of the user using a temperature sensor and a hyperspectral sensor. The processor may be configured to update a light intensity of a light source based on the temperature and the hyper spectral signature. In an embodiment, the surface of the skin may be illuminated based on the updated light intensity of the light source. The processor may be configured to compute a blood glucose level using the temperature, the hyper spectral signature, and the electrical skin impedance. The processor may be configured to provide the computed blood glucose level to the user.

According to embodiments illustrated herein, a non-transitory computer-readable storage medium having stored thereon, a set of computer-executable instructions for causing a computer comprising one or more processors to perform steps comprising, determining an electrical skin impedance between a first point and a second point of a surface of skin of the user using a skin impedance sensor. In an embodiment, the electrical skin impedance is indicative of an opacity of the surface of the skin between the first point and the second point. The one or more processors may be configured to determine a temperature and a hyper spectral signature of the skin of the user using a temperature sensor and a hyperspectral sensor. The one or more processors may be configured to update a light intensity of a light source based on the temperature and the hyperspectral signature. In an embodiment, the surface of the skin is illuminated based on the updated light intensity of the light source. The one or more processors may be configured to computing a blood glucose level using the temperature, the hyper spectral signature, and the electrical skin impedance. The one or more processors may be configured to provide the computed blood glucose level to the user.

### BRIEF DESCRIPTION OF THE DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 is a block diagram that illustrates a system environment in which various embodiments of the method and the system may be implemented;

FIG. 2 is a block diagram that illustrates a glucose monitoring device configured to non-invasively monitor a blood glucose level of a user, in accordance with some embodiments of the present disclosure;

FIG. 3 is a flowchart illustrating a method for monitoring the blood glucose level in a non-invasive manner, in accordance with some embodiments of the present disclosure; and

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

The present disclosure may be best understood with reference to the detailed figures and description set forth herein. Various embodiments are discussed below with reference to the figures. However, those skilled in the art will readily appreciate that the detailed descriptions given herein with respect to the figures are simply for explanatory purposes as the methods and systems may extend beyond the described embodiments. For example, the teachings presented and the needs of a particular application may yield multiple alternative and suitable approaches to implement the functionality of any detail described herein. Therefore, any approach may extend beyond the particular implementation choices in the following embodiments described and shown.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

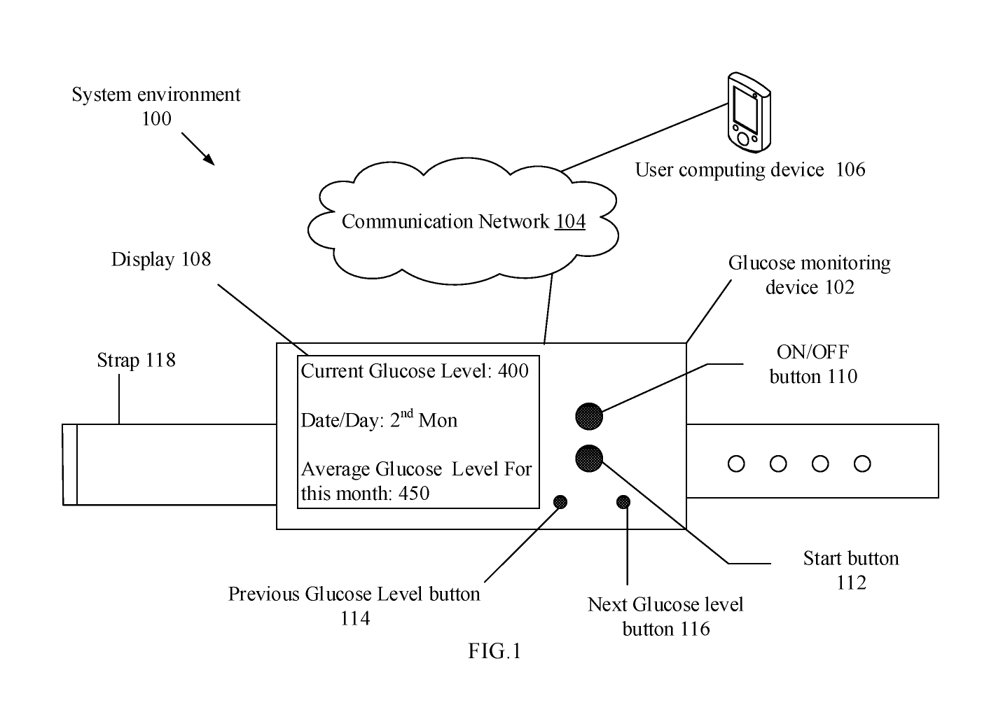

FIG. 1 is a block diagram that illustrates a system environment 100 in which various embodiments of the method and the glucose monitoring device 102 may be implemented. The system environment 100 may include a glucose monitoring device 102, a communication network 104, and a user computing device 106. In an embodiment, the glucose monitoring device 102 may communicate with the user computing device 106, via the communication network 104. In an embodiment, the glucose monitoring device 102 and the user computing device 106 may communicate with each other using one or more protocols such as, but not limited to, Open Database Connectivity (ODBC) protocol and Java Database Connectivity (JDBC) protocol. The glucose monitoring device 102 may further include a display 108, an ON/OFF button 110, a start button 112, a previous glucose level button 114, a next glucose level button 116, and a strap 118. In an embodiment, the ON/OFF button 110 may power ON the glucose monitoring device 102 and power OFF the glucose monitoring device 102 after an operation.

In an embodiment, the glucose monitoring device 102 may be a wearable device, such as a wrist watch. After the user wears the wearable glucose monitoring device 102 by tightening the strap 118 on a user's hand, the start button 112 may initiate the method for monitoring the blood glucose level of the user. In an embodiment, the display 108 may display the current blood glucose level of the user along with the current day and the current date. In an embodiment, the display 108 may further display an average glucose level of the user computed for a period of 30 days. Further, the previous glucose level button 114 and the next glucose level button 116 may enable the user to navigate between the historical blood glucose levels monitored by the glucose monitoring device 102.

In an embodiment, the glucose monitoring device 102 may refer to a computing device or a software framework hosting an application or a software service. In an embodiment, the glucose monitoring device 102 may be implemented to execute procedures such as, but not limited to, programs, routines, or scripts stored in one or more memories for supporting the hosted application or the software service. In an embodiment, the hosted application or the software service may be configured to perform one or more predetermined operations. The glucose monitoring device 102 may be realized through various types of servers such as, but are not limited to, a Java application server, a .NET framework application server, a Base4 application server, a PHP framework application server, or any other application server framework.

In an embodiment, the glucose monitoring device 102 may be configured to determine an electrical skin impedance between a first point and a second point of a surface of skin of the user using a skin impedance sensor (not shown). In an embodiment, the electrical skin impedance may be indicative of an opacity of the surface of the skin between the first point and the second point. The glucose monitoring device 102 may be configured to determine a temperature and a hyper spectral signature of the skin of the user using a temperature sensor (not shown) and a hyperspectral sensor (not shown). In an embodiment, the glucose monitoring device 102 may be configured to update a light intensity of a light source based on the temperature and the hyper spectral signature. In an embodiment, the surface of the skin may be illuminated based on an updated light intensity of the light source. The glucose monitoring device 102 may be configured to compute the blood glucose level using the temperature, the hyper spectral signature, and the electrical skin impedance. The glucose monitoring device 102 may be configured to provide the computed blood glucose level to the user.

In an embodiment, the user-computing device 106 may refer to a computing device used by the user. The user-computing device 106 may include one or more processors and one or more memories. The one or more memories may include computer readable code that may be executable by the one or more processors to perform predetermined operations. In an embodiment, the user-computing device 106 may present a user-interface to the user to transmit a request to monitor the blood glucose level. In an embodiment, the user-computing device 106 may be configured to receive the blood glucose level monitored by the glucose monitoring device 102. Further, user-computing device 106 may display the received blood glucose level to the user. Examples of the user-computing device 106 may include, but are not limited to, a personal computer, a laptop, a personal digital assistant (PDA), a mobile device, a tablet, or any other computing device.

A person having ordinary skill in the art will appreciate that the scope of the disclosure is not limited to realizing the glucose monitoring device 102 and the user-computing device 106 as separate entities. In an embodiment, the glucose monitoring device 102 may be realized as an application program installed on and/or running on the user-computing device 106 without departing from the scope of the disclosure.

In an embodiment, the communication network 104 may correspond to a communication medium through which the glucose monitoring device 102, and the user-computing device 106 may communicate with each other. Such a communication may be performed, in accordance with various wired and wireless communication protocols. Examples of such wired and wireless communication protocols include, but are not limited to, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), IEEE 802.11, 802.16, 2G, 3G, 4G, 5G cellular communication protocols, and/or Bluetooth (BT) communication protocols. The communication network 108 may include, but is not limited to, the Internet, a cloud network, a Wireless Fidelity (Wi-Fi) network, a Wireless Local Area Network (WLAN), a Local Area Network (LAN), a telephone line (POTS), and/or a Metropolitan Area Network (MAN).

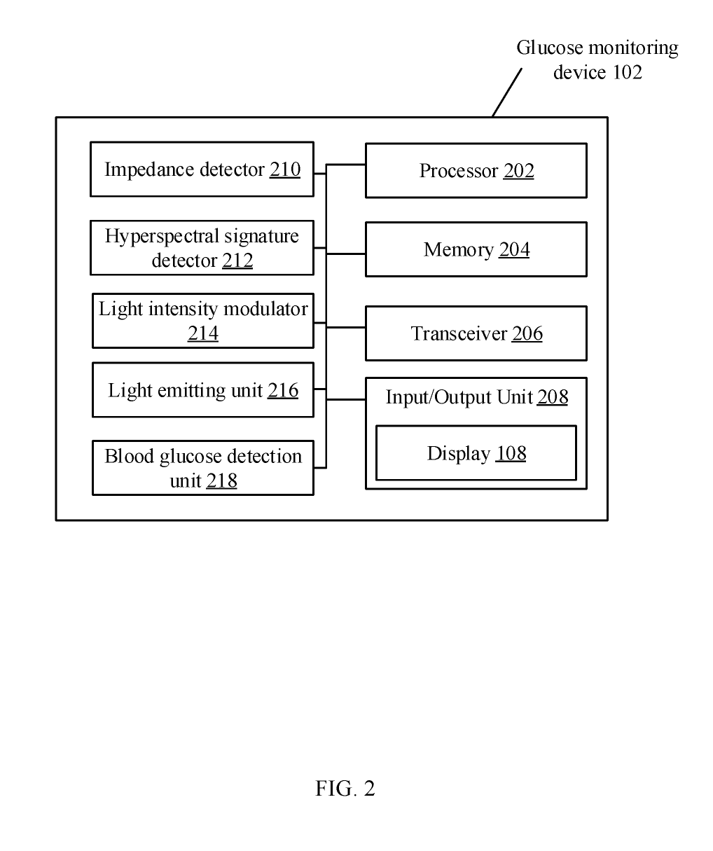

FIG. 2 is a block diagram that illustrates a glucose monitoring device configured to non-invasively monitor a blood glucose level of a user, in accordance with some embodiments of the present disclosure.

The glucose monitoring device 102 may include a processor 202, a memory 204, a transceiver 206, an input/output unit 208, an impedance detector 210, a hyperspectral signature detector 212, a light intensity modulator 214 and a light emitting unit 216. The processor 202 may be communicatively coupled to the memory 204, the transceiver 206, and the input/output unit 208, the impedance detector 210, the hyperspectral signature detector 212, the light intensity modulator 214, the light emitting unit 216, and the blood glucose detection unit 218.

The processor 202 may include suitable logic, circuitry, interfaces, and/or code that may be configured to execute a set of instructions stored in the memory 204. The processor 202 may be implemented based on a number of processor technologies known in the art. Examples of the processor 202 include, but not limited to, an X86-based processor, a Reduced Instruction Set Computing (RISC) processor, an Application-Specific Integrated Circuit (ASIC) processor, a Complex Instruction Set Computing (CISC) processor, and/or other processor.

The memory 204 may include suitable logic, circuitry, interfaces, and/or code that may be configured to store the set of instructions, which may be executed by the processor 202. In an embodiment, the memory 204 may be configured to store one or more programs, routines, or scripts that may be executed in coordination with the processor 202. The memory 204 may be implemented based on a Random Access Memory (RAM), a Read-Only Memory (ROM), a Hard Disk Drive (HDD), a storage server, and/or a Secure Digital (SD) card.

The transceiver 206 may include of suitable logic, circuitry, interfaces, and/or code that may be configured to receive a request for monitoring blood glucose level by initiating the start button 112 of the user's glucose monitoring device 102. The transceiver 206 may further be configured to receive a request from the user computing device 106 to compute the blood glucose level of the user. In response to the received request, the transceiver 206 may further be configured to transmit the computed blood glucose level to the user computing device 106. In an embodiment, the transceiver 206 may be further configured to transmit at least one of the electrical skin impedance, the temperature, the hyperspectral signature, and the blood glucose level to the user-computing device 106. In an embodiment, the transceiver 206 may be configured to receive one or more control signals transmitted by the user computing device 106.

The transceiver 206 may implement one or more known technologies to support wired or wireless communication with the communication network. In an embodiment, the transceiver 206 may include, but is not limited to, an antenna, a radio frequency (RF) transceiver, one or more amplifiers, a tuner, one or more oscillators, a digital signal processor, a Universal Serial Bus (USB) device, a coder-decoder (CODEC) chipset, a subscriber identity module (SIM) card, and/or a local buffer. The transceiver 206 may communicate via wireless communication with networks, such as the Internet, an Intranet and/or a wireless network, such as a cellular telephone network, a wireless local area network (LAN) and/or a metropolitan area network (MAN). The wireless communication may use any of a plurality of communication standards, protocols and technologies, such as: Global System for Mobile Communications (GSM), Enhanced Data GSM Environment (EDGE), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email, instant messaging, and/or Short Message Service (SMS).

The Input/Output (I/O) unit 208 may include suitable logic, circuitry, interfaces, and/or code that may be configured to receive an input or transmit an output. The input/output unit 208 may include various input and output devices that are configured to communicate with the processor 202. The I/O unit 208 may further include the display 108. The display 108 may be configured to display a reading pertaining to blood glucose level monitored by the glucose monitoring unit 102. Examples of the input devices include, but are not limited to, a keyboard, a mouse, a joystick, a touch screen, a microphone, and/or a docking station. Examples of the output devices include, but are not limited to, a display screen and/or a speaker.

The impedance detector 210 may include suitable logic, circuitry, sensors, interfaces, and/or code that may be configured to determine the electrical skin impedance between the first point and the second point of the surface of skin of the user using the skin impedance sensor. In an embodiment, the electrical skin impedance may be indicative of the opacity of the surface of the skin between the first point and the second point.

The hyperspectral signature detector 212 may include suitable logic, circuitry, interfaces, and/or code that may be configured to acquire the hyper spectral signature and the temperature of the skin surface. The hyperspectral signature detector 212 may house at least one or more sensors. In an embodiment, the one or more sensors may be a temperature sensor to measure the temperature and a hyperspectral signature sensor to measure the hyperspectral signature.

The light intensity modulator 214 may include suitable logic, circuitry, interfaces, and/or code that may be configured to update the light intensity of a light source based on the temperature and the hyper spectral signature. In an embodiment, the surface of the skin may be illuminated based on the updated light intensity of the light source. The blood glucose detection unit 218 may include suitable logic, circuitry, interfaces, and/or code that may be configured to compute a blood glucose level using the temperature, the hyper spectral signature, and the electrical skin impedance.

In operation, the glucose monitoring device 102 may be powered on using the ON/OFF button 110. After the glucose monitoring device 102 may be powered ON, the glucose monitoring device 102 may take a ten second time period to start the operation of monitoring the blood glucose level. In an embodiment, the glucose monitoring device 102 may be a portable device which can be worn on a wrist like a wrist watch. The portable device may not be restricted to a wrist watch or a wrist band.

In an embodiment, the start button 112 may initiate the method for monitoring the blood glucose level of the user. In an embodiment, the user computing device 102 may transmit a request for monitoring the blood glucose level to the glucose monitoring device 102. For example, the user computing device 106 may remotely power ON the glucose monitoring device 102 and start the operation of monitoring blood glucose level.

In response to the received request, the impedance detector 210 may determine the electrical skin impedance between the first point and the second point of the surface of skin of the user using the skin impedance sensor. In an embodiment, the electrical skin impedance may be indicative of the opacity of the surface of the skin between the first point and the second point. For example, when the glucose monitoring device may be worn on a hand, two terminals T1 and T2 of the glucose monitoring device 102 may come in contact with the skin of the user. In an embodiment, T1 is the first point of contact and T2 is a second point of contact. The skin impedance Z can be determined by a skin impedance sensor between the points T1 and T2. If an impedance Z is within a pre-determined value, then the skin touch is detected. The range of the skin impedance Z may be determined by the distance between the electrodes. The pre-determined range of the impedance value Z can also be set based on analyzing a number of samples employed on different types of skin.

The hyperspectral signature detector 212 may determine the temperature and the hyper spectral signature of the skin of the user. For example, the hyperspectral signature and the temperature are detected by the hyperspectral sensor and the temperature sensor, respectively. The hyperspectral sensor and temperature sensor may be embedded in the hyperspectral signature detector 212.

The light emitting unit 216 may be used to illuminate the skin surface. In an embodiment, the light emitting unit 216 may be an LED (Light Emitting Diode) light source. The light intensity modulator 214 may update the light intensity of the light source based on the temperature and the hyperspectral signature. In an embodiment, the surface of the skin is illuminated based on the updated light intensity of the light source. For example, the updation of light intensity may be considered as the stabilization of the power of the light source. If the values of temperature T and the electrical skin impedance Z are within a pre-determined value range, the power output of the light source may then take a value. This value of the light intensity is obtained iteratively by a pre-trained machine learning regression model M, using the variables, temperature T and electrical skin impedance Z, until the updated temperature and an updated hyper spectral signature is within a pre-defined range. The power of the light source is a function of temperature be w(t), the hyper spectral signature as a function of time is h(t) and impedance as a function of time is z(t), then total power output of the light source may be determined by

w(t)=M(h(t),z(t))

The regression model M may be trained with sufficient training samples obtained from a large number of subjects at different skin temperatures and skin thickness. The step of stabilization of the power of the light source is repeated at least three to four times or a pre-defined number of times, such that the change in power, that is w(t)−w(t−1) in between iteration, converges to a value in the order of 10̂-2 Watts.

The blood glucose detecting unit 218 may compute the blood glucose level using the temperature, the hyper spectral signature, and the electrical skin impedance. The blood glucose detecting unit 218 may then provide the monitored blood glucose level to the user. For example, the pre-trained machine learning regression model M is used to predict blood glucose level g.

Once, the values of hyperspectral signature, electrical skin impedance and skin temperature are obtained, the glucose g can be

g(t)=M(h(t),z(t),k(t)).

In an embodiment, the regression model M may be trained with different blood glucose levels, obtained from a number of iterations. The steps may be repeated for 10 seconds to come up with a time average determined blood glucose level.

In an embodiment, the glucose monitoring device 102 may show the average blood glucose level monitored during previous usage of the glucose monitoring device 102. For example, the previous glucose level 114 is a button which shows the average blood glucose level on a 30-day monthly basis. The next glucose level 116 is a button which may direct the user to the subsequent blood glucose levels monitored.

TABLE 1

| | Average Blood Glucose level in |
|---|---|
| Month | milli moles per liter (mmol/L) |
| January | 3.9 |
| February | 4.23 |
| March | 4.00 |
| April | 3.87 |
| May | 3.67 |

Table 1 shows the average blood glucose levels in units of milli moles per liter (mmol/L) on a monthly basis. If the current month is May, then the user may refer to the average blood glucose level of the previous months using the button, previous glucose level 114. To skip to the subsequent months and to the current month, the button next glucose level 116 may be used.

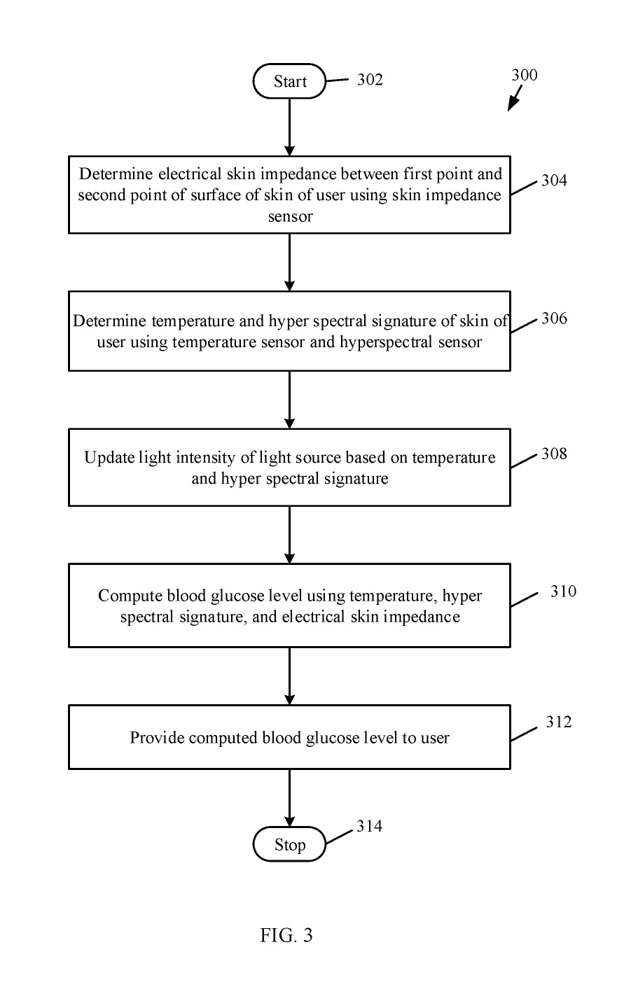

FIG. 3 is a flowchart illustrating a method 300 for monitoring the blood glucose level, in accordance with some embodiments of the present disclosure. The method starts at step 302 and proceeds to step 304.

At step 304, the glucose monitoring device 102 may be configured to determine the electrical skin impedance between the first point and the second point of a surface of skin of the user using the skin impedance sensor. In an embodiment, the electrical skin impedance may be indicative of an opacity of the surface of the skin between the first point and the second point. At step 306, the glucose monitoring device 102 may be configured to determine the temperature and the hyper spectral signature of the skin of the user using the temperature sensor and the hyperspectral sensor. At step 308, the glucose monitoring device 102 may be configured to update the light intensity of the light source based on the temperature and the hyper spectral signature. In an embodiment, the surface of the skin may be illuminated based on the updated light intensity of the light source. At step 310, the glucose monitoring device 102 may be configured to compute the blood glucose level using the temperature, the hyper spectral signature, and the electrical skin impedance. At step 312, the glucose monitoring device 102 may be configured to provide the computed blood glucose level to the user. Control passes to end step 314.

### Advantages of the Invention

The glucose monitoring device 102 may have the following advantages.

1. The glucose monitoring device 102 has adaptive properties, as it may handle differences in various skin types and skin thickness, moisture level on the skin, skin temperature, which may cause a change in the spectral signature.

2. The glucose monitoring device 102 may be configured to negate wrong sensor readings by pre-configuring an optimum threshold range for temperature and hyperspectral signature. It may further employ additional sensors and may prevent analysis of attenuated or saturated signal.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise. The terms “including”, “comprising”, “having” and variations thereof mean “including but not limited to”, unless expressly specified otherwise. The terms “a”, “an” and “the” mean “one or more”, unless expressly specified otherwise.

A description of an embodiment with several components in communication with each other does not imply that all such components are required. On the contrary, a variety of optional components are described to illustrate the wide variety of possible embodiments of the invention.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

The present disclosure may be realized in hardware, or a combination of hardware and software. The present disclosure may be realized in a centralized fashion, in at least one computer system, or in a distributed fashion, where different elements may be spread across several interconnected computer systems. A computer system or other apparatus adapted for carrying out the methods described herein may be suited. A combination of hardware and software may be a general-purpose computer system with a computer program that, when loaded and executed, may control the computer system such that it carries out the methods described herein. The present disclosure may be realized in hardware that comprises a portion of an integrated circuit that also performs other functions.

A person with ordinary skills in the art will appreciate that the systems, modules, and sub-modules have been illustrated and explained to serve as examples and should not be considered limiting in any manner. It will be further appreciated that the variants of the above disclosed system elements, modules, and other features and functions, or alternatives thereof, may be combined to create other different systems or applications.

Those skilled in the art will appreciate that any of the aforementioned steps and/or system modules may be suitably replaced, reordered, or removed, and additional steps and/or system modules may be inserted, depending on the needs of a particular application. In addition, the systems of the aforementioned embodiments may be implemented using a wide variety of suitable processes and system modules, and are not limited to any particular computer hardware, software, middleware, firmware, microcode, and the like. The claims can encompass embodiments for hardware and software, or a combination thereof.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A method for non-invasive monitoring of a blood glucose level of a user, the method comprising:
determining, by a glucose monitoring device, an electrical skin impedance between a first point and a second point of a surface of skin of the user using a skin impedance sensor, wherein the electrical skin impedance is indicative of an opacity of the surface of the skin between the first point and the second point;
determining, by the glucose monitoring device, a temperature and a hyper spectral signature of the skin of the user using a temperature sensor and a hyperspectral sensor;
updating, by the glucose monitoring device, a light intensity of a light source based on the temperature and the hyperspectral signature, wherein the surface of the skin is illuminated based on the updated light intensity of the light source;
computing, by the glucose monitoring device, a blood glucose level using the temperature, the hyper spectral signature, and the electrical skin impedance; and
providing, by the glucose monitoring device, the computed blood glucose level to the user.

2. The method of claim 1, wherein the updation of the light intensity of the light source is based on a pre-trained machine learning regression model.

3. The method of claim 1, further comprising determining an updated temperature and an updated hyper spectral signature of the skin of the user after the surface of the skin is illuminated based on the updated light intensity of the light source.

4. The method of claim 3, wherein the updation of the light intensity of the light source is performed iteratively until the updated temperature and the updated hyper spectral signature is within a pre-defined range.

5. The method of claim 1, wherein an average blood glucose level is determined based on a number of historical data of the blood glucose level.

6. The method of claim 1, wherein the electrical skin impedance is utilized to detect a skin touch.

7. The method of claim 1, further comprising transmitting at least one of the electrical skin impedance, the temperature, the hyperspectral signature, and the computed blood glucose level to a user-computing device, wherein the user-computing device transmits one or more control signals to the glucose monitoring device.

8. The method of claim 7, wherein the user-computing device performs one or more operations comprising running data acquisition, stabilization of the hyperspectral signature and analyzing spectral algorithm.

9. A glucose monitoring device to monitor a blood glucose level of a user, the glucose monitoring device comprising:
a processor; and
a memory communicatively coupled to the processor, wherein the memory stores
processor instructions, which, on execution, causes the processor to:
determine an electrical skin impedance between a first point and a second point of a surface of skin of the user using a skin impedance sensor, wherein the electrical skin impedance is indicative of an opacity of the surface of the skin between the first point and the second point;
determine a temperature and a hyper spectral signature of the skin of the user using a temperature sensor and a hyperspectral sensor;
update a light intensity of a light source based on the temperature and the hyper spectral signature, wherein the surface of the skin is illuminated based on the updated light intensity of the light source;
compute a blood glucose level using the temperature, the hyper spectral signature, and the electrical skin impedance; and
provide the computed blood glucose level to the user.

10. The glucose monitoring device of claim 9, wherein the processor is further configured to
update the light intensity of the light source is based on a pre-trained machine learning regression model.

11. The glucose monitoring device of claim 9, wherein the processor is further configured to determine an updated temperature and an updated hyper spectral signature of the skin of the user after the surface of the skin is illuminated based on the updated light intensity of the light source.

12. The glucose monitoring device of claim 11, wherein the processor is further configured to update the light intensity of the light source iteratively until the updated temperature and the updated hyper spectral signature is within a pre-defined range.

13. The glucose monitoring device of claim 9, wherein the processor is further configured to determine an average blood glucose level based on a number of historical data of the blood glucose level.

14. The glucose monitoring device of claim 9, wherein the processor is further configured to utilize the electrical skin impedance to detect a skin touch.

15. The glucose monitoring device of claim 9, wherein the processor is further configured to transmit at least one of the electrical skin impedance, the temperature, the hyperspectral signature, and the computed blood glucose level to a user-computing device, wherein the user-computing device transmits one or more control signals to the glucose monitoring device.

16. The glucose monitoring device of claim 15, wherein the user-computing device performs one or more operations comprising running data acquisition, stabilization of the hyperspectral signature and analyzing spectral algorithm.

17. A non-transitory computer-readable storage medium having stored thereon, a set of computer-executable instructions for causing a computer comprising one or more processors to perform steps comprising:
determining an electrical skin impedance between a first point and a second point of a surface of skin of the user using a skin impedance sensor, wherein the electrical skin impedance is indicative of an opacity of the surface of the skin between the first point and the second point;
determining a temperature and a hyper spectral signature of the skin of the user using a temperature sensor and a hyperspectral sensor;
updating a light intensity of a light source based on the temperature and the hyperspectral signature, wherein the surface of the skin is illuminated based on the updated light intensity of the light source;
computing a blood glucose level using the temperature, the hyper spectral signature, and the electrical skin impedance; and
providing the computed blood glucose level to the user.
